(19) United States
(12) Patent Application Publication
(10) Pub. No.: US 2022/0133145 A1
(43) Pub. Date: May 5, 2022

(54) MAGNETIC RESONANCE IMAGING METHOD AND DEVICE

(71) Applicants: CENTRE HOSPITALIER UNIVERSITAIRE VAUDOIS (CHUV), Lausanne (CH); FONDATION ASILE DES AVEUGLES, Lausanne (CH)

(72) Inventors: Benedetta FRANCESCHIELLO, Lausanne (CH); Lorenzo DI SOPRA, Lausanne (CH); Josefina Adriana Maria BASTIAANSEN, Lausanne (CH); Matthias STUBER, Romanel sur Lausanne (CH); Micah MURRAY, Lausanne (CH); Jerome YERLY, Charmey (CH)

(21) Appl. No.: 17/436,410

(22) PCT Filed: Mar. 5, 2020
(86) PCT No.: PCT/EP2020/055908

§ 371 Date: Sep. 3, 2021

(30) Foreign Application Priority Data
Mar. 5, 2019 (EP) 19160832.2

(51) Int. Cl. A61B 5/00; G01R 33/48; G01R 33/56; G01R 33/563; G01R 33/567; G01R 33/561; A61B 3/113; A61B 5/055

(52) U.S. Cl. CPC A61B 5/004; G01R 33/4826; G01R 33/5608; A61B 5/055; G01R 33/5673; G01R 33/5616; A61B 3/113; G01R 33/56391

(57) ABSTRACT

The present invention relates to a magnetic resonance eye imaging method, wherein an eye image is obtained from magnetic resonance image data acquired while the eye is moving, comprising determining eye orientation information data during magnetic resonance image data acquisition; binning the acquired magnetic resonance image data into groups according to eye orientation information data; and constructing a magnetic resonance image eye image from a selection of groups of magnetic resonance image data.

The present invention relates to magnetic resonance imaging.

Magnetic resonance imaging is broadly established as a medical imaging technique used in radiology. Where magnetic resonance imaging is used for medical diagnosis, the patient is positioned within an MRI scanner applying a very strong magnetic field around the area to be imaged. In the strong magnetic field, spins of certain atomic nuclei will align in an energetically favorable manner relative to the field. It is possible to alter this alignment by excitation with suitable radio frequency pulses; this in turn can be detected using suitable antennas.

As the strength of the detected signal depends inter alia on the different atoms in a given volume and their density, different substances such as water, fat, bones and so forth will produce different responses. Thus, with appropriate radio frequency excitation pulses and suitable arrangements of antennas in proximity to the body part examined, it is possible to construct three-dimensional images from within the body without using x-rays or other ionizing radiation.

However, it will be understood that despite significant progress made over time, known magnetic resonance imaging methods can still be improved as various problems currently exist.

For example, despite the development of improved electro magnetic pulse sequences, the excitation sequences used in magnetic resonance imaging often may not be used in their entirety. One of the reasons for this is that the object of which an image is to be provided is moving. As in conventional photography, this may result in blurred images. Thus, where e.g. images of the heart are needed for cardiologic examinations, the images would be blurred due to the beating of the heart. Accordingly, the beating of the heart needs to be taken into account; to this end, it has been suggested to acquire not only MRI data but to also acquire electrocardiogram (ECG) data simultaneously to the MRI data. From the ECG signals, periods within the cyclic beating of the heart can be identified where there is but little movement of the heart. Accordingly, images reconstructed from signals only relating to such periods are blurred to a lesser degree. This is known as ECG gating.

It has also been suggested that when imaging the heart, problems may occur not only due to the beating of the heart but also due to the patient breathing, resulting in additional motion. Therefore, it has been suggested to take into account both cardiac and respiratory phases. Reference is made inter alia to the paper “5D-whole-heart sparse MRI” by Lee Feng et al. in Magnetic resonance in medicine 79:826-838 (2018).

However, methods developed for heart imaging cannot be applied to other fields where different problems are given. Also, while gating may contribute to increased sharpness of the images obtained, the time needed for acquiring MRI data increases. Therefore, in order to acquire the MRI data, the patient must spend a rather long time in the MRI device. As very strong magnetic fields are needed for magnetic resonance imaging, and as strong magnetic fields can only be obtained over a rather restricted volume, it is necessary to place the patient inside a rather narrow tube. This in turn is considered uncomfortable by a large number of patients, particular where a prolonged period is necessary to acquire the MRI data. Accordingly, the prolonged acquisition periods may be a cause of discomfort of a patient. Then, prolonged data acquisition periods are not only uncomfortable to a patient but they are also expensive, and may lower the diagnostic yield as patient motion secondary to patient discomfort is more likely to occur during prolonged examination times.

Accordingly, other techniques are needed in other areas. With respect to the lung anatomy and pulmonary ventilation it has been suggested that these can be simultaneously evaluated, cf. J. Magn. Reson. Imaging 2019; 49: 411-422. In this paper, it is stated that certain sequences are particularly effective for imaging of the lung structure. It is also suggested to identify 4 or 6 respiratory phases, respectively, and to use them for binning the acquired MRI data. It is stated that the binning process suggested there is driven by the amount of data contained in each bin and that the bin widths are not constant. Furthermore, it is noted that an amount of motion is included in each bin and that this amount is variable within the same subject. Image reconstruction is then performed on all binned data sets.

While identifying relevant movement phases might help for certain applications, difficulties still remain in a large number of cases.

While the beating of the heart and respiration basically are cyclic, this does not apply for all movements of organs or other parts of the human body. Accordingly, it is not possible to identify phases of a cycle where motion is less regular.

Then, while generally a high resolution is desirable for any MRI image, there frequently exist particularly fine structures, resulting in even more significant problems of data acquisition. For such fine structures, not only is a prolonged period of MRI raw data acquisition necessary, but also minute movements of the body, object or organ examined constitute a particular and often severe problem.

This is of particular importance when patients such as small children are examined. In small children, the structures to be resolved generally are smaller than a comparable structure of an adult patient, but the children cannot be expected to be as cooperative as an adult patient. That frequently necessitates to use sedatives or anesthesia even where only an image is needed.

Problems may be observed with patients having a tremor or the like resulting in a lack of control of body movements. Where it is not possible to mechanically fix the body in the area examined, other measures are needed. A particular problem exists with respect to the eye. While it would be at least theoretically possible to mechanically fix an arm or leg, this is generally not ethically justifiable with respect to an eyeball. Furthermore, even where the patient is willing to cooperate, involuntary movements of the eye may still occur frequently. The movement of the eye generally is not cyclic, the movement can be very fast and the structures within the eye that need to be resolved in typical MRI applications are small. Thus, conventional gating techniques are insufficient. It is noted that some authors of papers relating to magnetic resonance eye imaging suggest to not only anesthetize a patient, but to also paralyze the eye. While feasible, such procedures should be avoided or at least minimized whenever possible. It should also be noted that in certain studies in animal models, it has been suggested to inject contrast enhancing substances, such as manganese, into specific parts of the eye. However, this is not readily feasible in humans without significant risks, including toxicity.

In a paper entitled “Dynamic Imaging of The Eye, Optic Nerve And Extra-Ocular Muscles With Golden Angle Radial MRI” by S. Sengupta, Albert et al., Invest ophtalmol viz sci 207: 58: 4010-4018. DOI: 10.1167/IOVS.17-21861, it has been stated that radiological imaging techniques, especially magnetic resonance imaging, (MRI) can provide a detailed anatomical information, but MRI has been used mostly in the static eye. It has been stated that detailed analysis of patient-specific extra-ocular muscle motions can be potentially useful in identifying exact etiologies in complex strabismus. The authors state that recent advances in the field of accelerated MRI have included the development of a technique known as “Golden Angle Radial Imaging” that has been used for several dynamic imaging applications including imaging of the heart, joints, abdominal organs and even human speech. The Golden Angle Radial Imaging method is considered to be known and established in the art. What is suggested in the paper is an analysis of motion patterns. To this end, landmarks that correspond to anatomic points of interest are manually identified in a subset of time series images, and then a time segment is started with all subjects looking in a specific first direction followed by sweeping the eyes to look into another direction and to then return to the original starting position. It is suggested that each cyclic eye movement can be estimated as an acute angle between segments connecting the lense with the optical nerve and that the length of the optic nerve in an image frame can be estimated by a polynomial fit over landmarked points. It is stated, however, that in an examination made, there was involuntary motion of the eye and optic nerve even in the resting state, even within 2 seconds. It is stated that data regarding positions, orientations, volumes and strains of specific anatomic structures can be extracted at much higher sampling rates than static MRI which typically requires at least about 100 to 200 ms per image according to Sengupta et al. It is stated that fast dynamic changes could be captured that might be missed by static gaze imaging and that a larger number of sample points can lead to much more well conditioned fits of parameters. It is suggested that a clinical application could be the evaluation of strabismus, where dynamic data might aid in pinpointing the exact extraocular muscles dysfunctions involved. It should be noted that Sengupta et al. in the context of eye MRI refers to Golden Angle sequences, but also states that a different fast imaging sequence commonly used in imaging moving anatomic structures would be steady-state free precession (SSFP) sequences having a temporal resolution that is high but still lower than that of the Golden Angle technique. Also, the Golden Angle method is stated to give a better contrast than SSFP in the soft tissue of the brain, but poorer CSF nerve contrast.

It will be noted that manually identifying sample points is disadvantageous. Furthermore, the short sampling time reduces resolution and promotes noise. Also, involuntary movements still seem to pose a problem.

In a review article entitled “Short Overview of MRI Artefacts” by L. J. Erasmus et al, SA Journal of Radiology, August 2004 pages 13 et sequ. a plurality of artefacts such as artefacts due the fat/water interface in the phase encoding or section-select directions that arise due to the difference in resonance of protons as a result of the micro magnetic environment are discussed and rectifying methods are suggested. Regarding motion artefacts, several methods are suggested, including patient immobilization, cardiac and respiratory gating, signal suppression of the tissue causing the artefact, choosing the shorter dimension of the matrix as the phase-encoding direction, view-ordering or phase-reordering methods and swapping phase and frequency-encoding directions to move the artefact out of the field of interest.

In a paper entitled “Three-Dimensional, in Vivo MRI With Self-Gating And Image Co-Registration in The Mouse” by B. J. Nieman et al, Magn. Reson. Med. 2009, May; 61(5): 1148-1157. DOI: 10.1002/MRN.2195, self-gated imaging methods and image co-registration for improving image quality in the presence of motion are suggested. Self-gated signal results from a modified 3D gradient-echo sequence are stated to show detection of periodic respiratory and cardiac motion in an adult mouse. It is stated that image quality during long high-resolution scans can be adversely affected by non-periodic, bulk rotations and translations of an embryo. Artefact due to such motion is stated to be not unique to mouse embryo imaging; studies of dynamic contrast enhancement and functional MRI (fMRI) are stated to require an exact orientation of serially-acquired images for proper analysis of intensities over a time series. It is stated that to ensure proper alignment, images could be registered together during post-processing to eliminate or limit the effects of motion in studies.

Regarding MRI sequences, in US 2017/0299678 A1, it has been stated that selectively exciting bulk protons in certain tissue components, e.g. water, while suppressing the excitation of others, e.g. fat, can lead to images with better contrast for desired features, providing binomial, off-resonance RF excitation pulses for differentiating tissue excitation that yields a larger fat suppression than prior art water excitation methods. It is stated that proper balancing of the frequency offset and the pulse duration with a relative phase offset between the pulses leads to large band-width pass and stop bands for water and fat, respectively. The pulses are stated to be applicable with short or even zero interpulse delay, leading to substantial time savings in an imaging sequence.

Reference is also made to a publication entitled “Flexible Water Excitation for Fat-Free MRI at 3T Using Lipid Insensitive Binominal Of-Resonant RF Excitation (Libre) Pulses” by J. A. M. Bastiaansen and M. Stuber. The authors suggest that while optimizations of a frequency offset and a pulse duration would be mandatory, fat suppression remains effective over a relatively large range of parameter settings.

In a paper entitled “Landmark Detection For Fusion of Fundus And MRI Towards a Patient-Specific Multi-Model Eye Model”, IEEE Transactions on Bio-medical Engineering Class Files, by S. DeZanet IEEE Transactions on Bio-Medical Engineering 62(2) September 2014; DOI: 10.1109/TBME. 2014.2359676, it is stated that retinoblastoma is a frequent eye cancer almost exclusively occurring in and affecting young children. It is stated that in treatments, it is important to monitor the progression of the tumor over time to assess the effectiveness, but that proper treatment planning is time consuming and error-prone due to the high work-load for the radio-therapist and that the analysis of the MRI is a tedious task in a three-dimensional volume. The authors suggest an automatic segmentation and fusion of two commonly-used diagnostic image modalities in retinoblastoma, namely fundus photography and MRI volumes. It is suggested to detect the eye centers using analysis of MRI images and to then segment the retinal surfaces to provide a surface for fundus projection. For this, inter alia, the optical axis has to be found, and specific algorithms are suggested to this end. However, to acquire the MRI data with reduced motion artefacts, the infant patients examined had to be anesthetized.

In a doctoral dissertation submitted to ETH Zurich (Dissertation no. 19765) by Marco Piccirelli, a method for dynamic imaging of an eye is disclosed. A T1-weighed turbo field echo pulse sequence is applied in this disclosure. This disclosure requires periodic and precise eye-movements synchronized with the MRI acquisition. If any unexpected movement occurs, the acquisition needs to be discarded.

Piccirelli et al. (2016) Proc. Intl. Soc. Mag. Reson. Med., volume 24, discloses high spatiotemporal resolution dynamic MRI imaging of the orbit during repetitive eye movement.

It is desirable to improve magnetic resonance imaging methods and magnetic resonance imaging devices. In particular, it would be desirable to improve the resolution and/or to reduce the acquisition time and/or to improve imaging despite movements of an object examined.

It is desirable to provide improved eye images by magnetic resonance eye imaging methods and devices and it would be desirable to reduce the strain and/or cost of MRI data acquisition.

Therefore, the object of the present invention is to provide improved methods for magnetic resonance imaging of an eye during movement of the eye. In other words, the eyes are moving while the scanner is acquiring the imaging data.

The independent claims indicate how this object can be achieved. Some of the preferred embodiments are claimed in dependent claims.

In a first aspect, the present invention relates to a magnetic resonance eye imaging method, wherein an eye image is obtained from magnetic resonance image data acquired while the eye is moving, comprising determining eye orientation information data during magnetic resonance image data acquisition, binning the acquired magnetic resonance image data into groups according to eye orientation information data; and constructing a magnetic resonance eye image from a selection of groups of magnetic resonance image data.

The present inventors have surprisingly found that the blur of the reconstructed images of an eye is significantly reduced if the image is reconstructed from MRI data collected for the same orientation of the eye. The method of the present invention provides means for binning the MRI data according to eye orientation information, and for determining eye orientation information data during MRI data acquisition. In contrast to the approaches of the prior art, acquisition of the data is uninterrupted and determined orientation of the eye during data acquisition is only used in post-processing, As demonstrated by the Example 2 and Reference Example 2, the so reconstructed images are significantly less blurred than the images reconstructed from the same amount of data collected consecutively.

According to a first basic idea, a magnetic resonance eye imaging method is suggested, wherein an eye image is obtained from MRI data acquired while the eye is moving, comprising determining eye orientation information data during MRI data acquisition; binning the acquired MRI data into groups according to eye orientation information data; and constructing an MRI eye image from a selection of groups of MRI data. Herein, a reconstructed MRI eye image is understood as 3D MRI image, unless otherwise stated.

Herein, binning of the acquired MRI data is understood as selecting acquired MRI data for processing together to reconstruct an MRI image, wherein the data do not need to be temporally collected at the same time or consecutively.

Accordingly, the present invention suggests that MIRI data of the object examined are obtained while the object is moving and to acquire additional information that relates not to the movement or movement cycle, but to the orientation of the object. As the object orientation data is determined in parallel to, but separate from, the actual MRI data acquisition, there is no need for a physician to manually identify landmarks in images constructed.

It is therefore noted that the present invention allows an orientation-resolved reconstruction of magnetic resonance images capturing an organ of interest while the organ is moving. It can be shown that eye orientations deduced from images constructed with 3D MIRI data binned and analyzed according to the invention correlate strongly with the orientation determined using an eye tracker. This clearly indicates that the binning suggested here leads to an imaging in orientations that closely correspond to correctly measured orientations.

This is of particular importance for the eye, as eye movements have been known to be important symptoms and thus candidate biomarkers e.g. for neurodevelopmental, psychiatric, cognitive and other disorders, including but not limited to dyslexia, autism, psychosis, and Alzheimer's disease. Despite the slow speed of MR image data acquisition relative to eye reorientation speeds, eye-orientation can be allowed and no anesthesia or sedation as is necessary in clinical protocols for retinoblastoma detection and/or fixation protocols with prescribed blinks as typically used in conventional research studies are necessary. Note that using a method wherein eye movement is allowed is extremely helpful in particular for clinical purposes as it allows inter alia to perform a motion-resolved image construction of the eye.

It should also be noted that the method of the invention inter alia is also extremely helpful for basic research purposes, particularly as naturalistic stimuli with which participants freely move their eyes are used more often in research and as the study of pediatric and geriatric populations become increasingly common. Moreover, eye position and movements are a useful indicator of participants' attention and arousal. Furthermore, robust non-invasive imaging of the eye that also allows a field of view including the brain will likely be a harbinger of insights regarding the links between eye and brain anatomical and functional organization, including domains such as ocular dominance and retinotopy (among others).

By binning the acquired MRI data into groups according to the orientation information data, it surprisingly has been found possible to reduce the overall blur of images. Note that examining the eye as an object is a particularly suitable and medically useful application of acquiring MRI data during movement and determining orientation information during MRI data acquisition.

On the one hand, this is due to the fact that the eye hardly changes its shape while it is moving. Although, when looking at an object, the eye may re-focus depending on the distance of the object, the overall change of shape generally is of little concern; this holds in particular in an MRI setting where the patient is lying inside the MRI magnet tube and any pattern within the patient's field of view will be at approximately the same distance. It is also noted that unless specific conditions are created, in a standard setting during MRI data acquisition, the light level need hardly change for mere anatomic imaging so that accordingly, not even the iris needs to change its aperture.

Therefore, the eyeball itself can basically be considered a rigid object, notwithstanding, of course, that the nerves leading to the retina will move while the eye is moving and that some muscles surrounding the eye will change their shape. Nonetheless, for the purpose of the invention, the eye can be considered a sufficiently rigid object, similar to for example a bone.

Note that it is possible to acquire Mill data even where the eye is refocusing and/or adapting to changing illuminations. It will be noted that for the eye, where the head of the patient is at rest and might, under specific circumstances even be fixated, only two degrees of freedom of the orientation angles need to be taken into account.

It is noted that large fractions of MRI raw data can be used for constructing magnetic resonance eye images for a given eye orientation. This is quite different from only taking into account those MRI data acquired during respiratory cycle phases where the movement of an organ is known to be in minimum and the “gating” only considers a tiny fraction of all data. In the present invention, the binning does not rely on a fully or almost complete absence of motion; rather, those parts of MRI data associable with a given orientation shall be used.

An MRI image constructed from data binned according to the present invention thus will relate to a specific orientation of the eye or a specific range of orientation. In this manner, there is no super-position of image information obtained with largely different eye orientations and hence, the overall MRI image can be significantly sharper than known in the prior art. This holds even where the eye was not at rest when passing through a given orientation.

In a further aspect of the present invention, the magnetic resonance image data are acquired with a free running magnetic resonance image and/or in a manner not triggered by an eye orientation determined.

In a further aspect of the present invention, the eye image is obtained from magnetic resonance image data acquired intermittent to or simultaneous with an eye motion.

In a further aspect of the present invention, determining eye orientation information data during magnetic resonance image data acquisition comprises tracking the orientation of the eye or the orientation of a surface related to the eye.

It will be obvious to the average skilled person that the magnetic resonance object imaging method of the present invention can produce object images with a variety of different MRI pulses or pulse sequences. It is well known in the art that different excitation pulses may be used for different purposes. For example, there frequently is a problem that the contrast of MRI images is too low as different tissues cannot be sufficiently distinguished. Therefore, it may be desirable to use an MRI sequence particularly suitable for obtaining appropriate images. It will be understood by a skilled person that specific requirements during acquisition might necessitate specific pulse sequences. For example, in one embodiment it might be necessary to obtain a particularly high resolution. In another embodiment, it might be necessary to better distinguish between certain tissues or material in the volume examined, for example in order to improve the contrast between fat and water. In a specific embodiment, it might be necessary to obtain MRI data particularly fast, for example because the patient needs to be assessed very quickly.

A number of different sequences or excitation pulses may be used for the present invention. It is possible to select a sequence that allow to obtain 2D images or to select a sequence that also allows to obtain a 3D images, which obviously is preferred. Furthermore, sequences can be selected such that signals from fat tissue are suppressed or such that signals from fat tissue are not suppressed. The sequence can be selected such that it corresponds to a Golden Angle sequence or could be selected such that this is not the case. Furthermore, the sequence might follow a radial, cartesian or spiral pattern. The sequence can e.g. be a bSSFP, GRE (gradient echo), EPI, TSE or GRASE sequence.

It will be understood that sequences can be selected such that different of the properties listed above of sequences can be simultaneously implemented. This is helpful as a sequence a physician is familiar with and which already is implemented on an MRI scanner available can be used. For example, a sequence could be used that is a 3D, fat suppressed, Golden Angle, radial GRE sequence, although any other combination may be used as well, and may be more or less useful for specific ophtalmologic purposes.

It is noted in particular that the method of the present invention allows to use uninterrupted sequences, for example an uninterrupted gradient recalled echo (GRE) sequence. Therefore, basically the continuous acquisition of the magnetic resonance imaging device during examination of a given patient is possible. It will be understood that in this manner the time spent for recording an image is reduced, increasing the overall comfort of the patient and reducing the costs of an MRI image due to the better utilization. Accordingly, it is considered advantageous to acquire data with a free running MRI or with sequences not triggered by an orientation of the object determined, that is not triggered by the patient looking into a specific direction. It is to be therefore noted that the imaging data acquisition is uninterrupted and independent of the eye movements. The eyes can move freely during the acquisition of data. This will help to reduce overall examination time and, given the high costs of an operating hour of an MRI, will reduce costs of an examination significantly.

Also note that as a free-running sequence, for example the free-running gradient recalled echo (GRE) sequence with 3D radial (spiral) Golden-Angle-Trajectory (phyllotaxis) can be used for uniform sampling of k-space, acquisition is straightforward and possible with known sequences and techniques such as fat-saturation, slab selection needed to increase specific tissue contrast can be implemented as needed with the method of the present invention.

It is also noted that once the initially acquired MRI data have been binned and images have been constructed for different orientations, it is possible to process any 3D orientation resolved anatomical images of the eye further. For example, it is possible to obtain orientation corrected 3D images by registration of orientation resolved volumes from the two-dimensionally binned 3D reconstruction. It is noted that the multidimensional reconstruction with parallel imaging and a compressed sensing framework exploits sparsity along the extra dimensions of orientation.

It will be understood that when acquiring MRI data with a free running MRI while the eye is changing its orientation, it is possible to record the MRI data together with sufficiently precise time stamps and to determine the eye orientation information data with corresponding time stamps. In this manner, it is possible to bin the acquired MRI data after acquisition. In a preferred embodiment, at least 4, preferably 5, 6, 7, 8, 9, 10 time steps per sequence and/or time steps not further apart than 1/10s, preferably not more than 1/24s, in particular not more than 1/30s apart are determined. Note that a high temporal resolution of the eye orientation allows interpolation of a current orientation.

In a further aspect of the present invention, determining eye orientation information data during magnetic resonance image data acquisition comprises causing the eye to orient in space according to a known pattern.

This may be helpful and important in cases where the patient is requested to observe a moving pattern, for example a point shown on a screen or projected onto the inner surface of the MRI tube within the field of view of the patient. When doing so, the pattern could be selected in a manner providing sufficient data for any given eye orientation of interest. For example, the point could move slowly within an upper left corner area, then move swiftly to the lower right corner and then move slowly in this area. It should be noted that where a pattern is to be followed, a plurality of possibilities exist to display or generate the pattern. A screen for displaying the pattern could be placed within the tube. Then, the patient could be asked to follow a pattern such as a point moving across the display. In another embodiment, an illumination point such as from a laser pointer could be projected onto the inner surface of the magnetic tube. Also, a number of fibers could be placed in the tube, with the fiber ends being spaced apart. During examination, light could be injected into varying fibers and the patient could be asked to look at the fiber currently illuminated. In an even more simple setup, optical marks could be provided on the inner surface of the tube, for example numbers 1-9 arranged on 3×3 grid. The patient could be asked to look at changing numbers. The patient could be asked to look at a given number at a given time using a conventional intercommunication system.

Therefore, it can be seen that the method of the present convention can be easily implemented even with already existing magnetic resonance image systems.

Nonetheless, cases may occur where the patient is not capable of following a projected moving pattern, for example because of involuntary movement of the eye due to a medical condition of the patient or to pharmaceuticals administered prior to the MRI examination. In such a case, it may be preferred to just determine the eye orientation during the acquisition and to then decide later on how a binning can be effected best.

It will be understood that binning the acquired MRI data into groups that are too small may result in a lack of detail due to the lack of MRI data considered whereas increasing the bins by increasing the range of orientations the bin refers to might result in a blurring of fine details due to the super-position of MRI data obtained for eye orientations that differ largely. Accordingly, it may be helpful to bin the acquired data according to a first binning, construct an MRI image, determine whether or not at least some 2D images in some planes and/or at some orientations are acceptable and/or of medical or diagnostic use and to re-iterate the binning and MRI eye image construction, if this should not be the case.

It will be understood that while using a large number of bins would be possible, the amount of data in each group or bin after a given acquisition time would then be reduced. Accordingly, it might become necessary to increase the acquisition time if the number of bins is too large. However, frequently, there is no need to depict the eye in a very large number of different orientations. Rather, it will frequently be sufficient to have a rather small number of different orientations, for example where a patient is to look up, down and/or to look left and right. Therefore, in a preferred embodiment, the number of bins in each direction (or rather for each orientation of the eye) can be rather small. For example, three, four or five ranges could be used along up/down directions and three, four or five bins could be used along left/right directions. Using a larger number across the entire field of view and/or along a specific line such as the edges of the field of view will not significantly improve resolution, sharpness and so forth; however, using a number too small will also not produce favorable results as the range of orientations considered in one bin would be too large.

Also, where a stimulation protocol is used, for example by showing a moving pattern to a patient, the time spent in specific orientations such as far left, far right, far up and far down, can be higher compared to time spent in other orientations, increasing the resolution for the more important orientations. This may be the case even where the actual pattern shown varies in a random manner.

Note that where a visual stimulation protocol is used, eye orientation related to the specific stimulation may be reconstructed, leading to for example the detection of anatomical impairments at the retinal optic nerve level in clinical applications. It should be noted that applications in neuropathology and neuroimaging exist. The method thus allows simultaneous and comprehensive investigations of both ocular and brain volumes both for clinical and for research purposes. In this way, the anatomic and functional integrity of the full visual pathway can be assessed. The present invention is particularly helpful because the necessary clinical examinations can now be conducted without an anesthetics or sedation in freely-behaving patients of all ages. Even where animals are examined rather than humans, it is possible to apply the method as a large number of animals will follow a pattern shown and/or can have their eyes tracked.

Note that even where an intermediate result obtained by some intermediate iteration might need to be judged by a medical practitioner such as a physician, it would also be possible to automatically detect whether or not fine details are present in an MRI image and/or whether the amount of MRI raw data binned into a given group can be judged to be sufficient.

Depending on the specific MRI, the specific MRI pulse sequence used and so forth, the overall amount of MRI data binned into a given group and/or necessary to obtain an MRI eye image may vary for a given purpose such as diagnostic purposes. Nonetheless, it is to be anticipated that respective thresholds of the data volume needed in a given group or bin can be estimated in a satisfying manner. This can even be done automatically.

In a preferred embodiment, time stamps are assigned to the MRI data while acquired such that for every pulse, a plurality of time stamps is co-recorded together with the signal detected in response to any excitation pulse used.

It is possible to determine the eye orientation information data either intermittent to or simultaneous with the MRI data acquired. In particular, it is possible to show a first pattern to a patient and ask him to look at the pattern, then generate one or a few MRI pulses, and then change the pattern shown so that the patient has to look in another direction. When this is done, it would be sufficient to change the pattern shown intermittently to the MRI data acquisition. However, generally it would be more preferable to simultaneously determine eye orientation information data while the MRI data are acquired, that is while MRI pulse sequences are generated.

It will be understood by the average skilled person that it is not preferred to trigger the MRI data acquisition in view of an object orientation, that is, for example, because the patient has been found to look at a given direction. If this is done, there would be periods where the MRI is not acquiring MRI data. However, what can be done is that the patient is shown a computer-generated pattern with a feature he is asked to follow with his eyes. The movement of the pattern or the feature could be triggered or synchronized with an MRI pulse sequence, ensuring for example that the pattern changes e.g. every n pulses with n=1, 2, 3, 4, 5, 6, 7, 8, 9 or 10.

Also, changes after a random number n could be effected. It will be understood that it may be preferable to have more than one MRI sequence for any given feature position shown to the patient so that any reorientation of the eye to follow a feature shown loses importance and weight vis-à-vis the overall acquisition time spent at a given position. However, if the time the feature resides at a given position becomes too long, it is likely that the patient will start to blink, or that his eyes moves involuntarily, even if the respective change of orientation is minimal. Therefore, it generally is preferred if the time span at any given orientation can be smaller than 30 seconds, preferably shorter than 20 seconds and in particular be shorter than 10 seconds. It is to be anticipated that a short time span is preferred. For a naturalistic setting, a saccadic eye movement app. four times a second would allowed. By showing a specific pattern the patient has to follow, the time between eye movements can be prolonged and hence, the time span can have a useful length. Note that a useful time span may vary for different patients.

It is also possible to show a continuously moving pattern to a patient. In this case, a number of bins can be defined corresponding to the path a feature shown to the patient takes. Note that in particular in such a case, interpolation is possible.

It will be understood that the determination of object orientation information during MRI data acquisition may be effected by one or both of tracking the actual orientation or by stipulating that the object is oriented in a specific manner, e.g. by showing a specific pattern to the patient It will be understood that that the determination of the orientation of the eye can be and preferably will be effected by such visual stimulation and that the visual stimulation will follow a specific protocol and/or by simultaneous tracking. It will be understood that any (photographic or videographic) image acquired for eye tracking purposes will not be static, so that a re-orientation of the eye will result in MRI data being binned into other groups, even when the re-orientation is fast.

In this context, it is noted that so-called eye trackers are well known that allow to determine the direction into which the person is looking. Basically, images of the eye and/or of the face are recorded and the direction a person is looking to is determined therefrom. Such photographic (or videographic) imaging for eye tracking purposes can be effected using conventional cameras. However, it should be noted that possibilities exist to operate a magnetic resonance imaging system without placing an eye-tracking camera or other complete eye tracking device inside the MR scanner. In particular, it is possible to use for example fiber-based optics for observing the patient and a current orientation of his eye, to use mirrors and the like. It will be obvious to the skilled person that once the direction a person is looking at is known, so will be the orientation of the eye.

Note that the techniques of tracking and of showing a pattern the patient has to follow can be combined, for example to determine whether or not or to what degree a patient actually is able to follow a pattern shown.

In a further aspect of the present invention, determining eye orientation information data during magnetic resonance image data acquisition comprises determination of eye orientation information data according to a two-dimensional pattern.

In a yet further aspect of the present invention, binning the acquired magnetic resonance image data into groups according to eye orientation information data comprises a two-dimensional binning.

In a preferred embodiment, the eye orientation information comprises a determination of object orientation information data according to a two-dimensional pattern. Thus, it can for example be determined whether the patient is looking left/right and up/down. Accordingly, in a typical set-up, the binning will be a two-dimensional binning according to the two-dimensional pattern. A simple two-dimensional binning is particularly useful where no problems exist with respect for example to strabismus. However, if the patient is unable to follow a pattern with both eyes simultaneously, it might be useful and/or necessary to actually track the orientation of each eye of the patient independently and to then use a two-dimensional binning for the left eye and a separate two-dimensional binning for the right eye. Basically, this would correspond to a four-dimensional binning; however, it is easily possible to double the data set and to then apply two-dimensional binning to the first set for the left eye orientation and to apply two-dimensional binning of the second/copied data set according to the right eye orientation. An image could then be constructed according to the binning and the two different images obtained for the first and second set could be combined so as to have the two different eyes looking into the same direction or almost the same direction.

It will be understood that in a preferred embodiment, the construction of the MRI image from a selection of groups of MRI data will result in a three-dimensional image having a number of planes. In other words, although reference is made to an MRI image, this does not restrict the invention to a two-dimensional image. Rather, three-dimensional volume information depicting a user is also referred to as being an “image”. Thus, existing techniques to provide imaging of a volume are applicable with the present invention.

Thus, in a further aspect of the present invention, constructing a magnetic resonance image from a selection of groups of magnetic resonance image data comprises constructing a 3D image having a number of planes.

Also, it is possible to construct sequences of images wherein the sequence is selected such that it corresponds to a sequence of (neighboring) orientations. Accordingly, the eye can be shown as if it were moving.

Thus, in a further aspect of the present invention, constructing a magnetic resonance eye image from a selection of groups of magnetic resonance image data comprises constructing a sequence of images constructed according to a sequence of orientations.

Note that in a typical application, it is not necessary to acquire MRI data only in a first orientation, then acquire MRI data in only a 2nd orientation, then acquire MRI data in the 3rd orientation and so forth, but that the MRI data can be acquired while the eye is changing its orientation. By then re-grouping the MRI data, a 3 dimensional representation of the eye can be obtained.

While the magnetic resonance eye image relates to imaging the eye, it will be understood by an average skilled person and/or a medical practitioner such as an ophthalmologist that it is useful to also provide images of the surrounding. Accordingly, the volume scanned typically is comprising not just the eye but additional volumes, for example the entire head.

Thus, in a further aspect of the present invention, a body part is scanned comprising the entire visceral cavity wherein the eye is located. The method of the invention relates further to an embodiment, wherein a body part is scanned comprising the entire visceral cavity wherein the eye is located, wherein the eye orientation is determined by a showing a pattern to be followed.

Protection is also sought for a magnetic resonance imaging system comprising an MRI data acquisition arrangement adapted to acquire MRI data from a region of interest including the eye and while the eye is moving, and an eye orientation information data determination arrangement adapted for determining eye orientation information data during MRI data acquisition in a manner allowing to assign an orientation of the eye to different parts of the MRI data. In particular, a display means for displaying a pattern to be tracked with the eyes and/or an eye-tracker can be provided.

In a preferred embodiment, the magnetic resonance eye imaging system will also comprise an image constructing arrangement adapted to bin the acquired MRI data into groups according to eye orientation information data; and to construct an MRI eye image from a selection of groups of MRI data.

Furthermore, it will be obvious that the binning and constructing of the actual image from the binned data will be computer-implemented. It is noted that this can be done remote from the actual MRI data acquisition system. Accordingly, protection is also sought for a magnetic resonance eye image construction arrangement for constructing eye images from magnetic resonance imaging data acquired during movement of the eye, the magnetic resonance eye image construction arrangement comprising an input for inputting MRI data acquired from a region of interest including the eye and while the eye is moving, and for inputting eye orientation information data relating to eye orientation information data determined during MRI data acquisition, and an image constructing arrangement adapted to bin the acquired MRI data into groups according to eye orientation information data; and to construct an MRI eye image from a selection of groups of MRI data.

Thus, in another aspect the present invention relates to a magnetic resonance eye imaging system, comprising a magnetic resonance image data acquisition arrangement adapted to acquire magnetic resonance image data from a region of interest including the eye and while the eye is moving, and an eye orientation information data determination arrangement adapted for determining eye orientation information data during magnetic resonance image data acquisition in a manner allowing to assign an orientation of the eye to different parts of the magnetic resonance image data.

In a further aspect, the magnetic resonance eye imaging system of the present invention relates to an embodiment further comprising an image constructing arrangement adapted to bin the acquired magnetic resonance image data into groups according to eye orientation information data; and to construct a magnetic resonance image eye image from a selection of groups of magnetic resonance image data.

In a further aspect, the present invention relates to a magnetic resonance eye image construction arrangement for constructing eye images from magnetic resonance imaging data acquired during movement of the eye, the eye image construction arrangement comprising an input for inputting magnetic resonance image data acquired from a region of interest including the eye and while the eye is moving, and for inputting eye orientation information data relating to eye orientation information data determined during magnetic resonance image data acquisition, and an image constructing arrangement adapted to bin the acquired magnetic resonance image data into groups according to eye orientation information data; and to construct a magnetic resonance image eye image from a selection of groups of magnetic resonance image data. Further aspects and/or embodiments of the present invention are disclosed in the following numbered items:
1. A magnetic resonance eye imaging method, wherein an eye image is obtained from magnetic resonance image data acquired while the eye is moving,
   comprising
   determining eye orientation information data during magnetic resonance image data acquisition;
   binning the acquired magnetic resonance image data into groups according to eye orientation information data;
   and
   constructing a magnetic resonance image eye image from a selection of groups of magnetic resonance image data.
2. A magnetic resonance eye imaging method according to the previous item, wherein the magnetic resonance image data are acquired with a free running magnetic resonance image and/or in a manner not triggered by an eye orientation determined.
3. A magnetic resonance eye imaging method according to one of the previous items, wherein the eye image is obtained from magnetic resonance image data acquired intermittent to or simultaneous with an eye motion.
4. A magnetic resonance eye imaging method according to one of the previous items, wherein determining eye orientation information data during magnetic resonance image data acquisition comprises tracking the orientation of the eye or the orientation of a surface related to the eye.
5. A magnetic resonance eye imaging method according to one of the previous items, wherein determining eye orientation information data during magnetic resonance image data acquisition comprises causing the eye to orient in space according to a known pattern.
6. A magnetic resonance eye imaging method according to one of the previous items wherein determining eye orientation information data during magnetic resonance image data acquisition comprises determination of eye orientation information data according to a two-dimensional pattern.
7. A magnetic resonance eye imaging method according to one of the previous items wherein binning the acquired magnetic resonance image data into groups according to eye orientation information data comprises a two-dimensional binning.
8. A magnetic resonance eye imaging method according to one of the previous claims wherein constructing a magnetic resonance image from a selection of groups of magnetic resonance image data comprises constructing a 3D image having a number of planes.
9. A magnetic resonance eye imaging method according to one of the previous items wherein constructing a magnetic resonance eye image from a selection of groups of magnetic resonance image data comprises constructing a sequence of images constructed according to a sequence of orientations.
10. A magnetic resonance eye imaging method according to one of the previous items wherein a body part is scanned comprising the entire visceral cavity wherein the eye is located.
11. A magnetic resonance eye imaging method according to the previous item wherein the eye orientation is determined by a showing a pattern to be followed.
12. A magnetic resonance eye imaging system,
   comprising
   a magnetic resonance image data acquisition arrangement adapted to acquire magnetic resonance image data from a region of interest including the eye and while the eye is moving,
      and
      an eye orientation information data determination arrangement
         adapted for determining eye orientation information data during magnetic resonance image data acquisition
         in a manner allowing to assign an orientation of the eye to different parts of the magnetic resonance image data.
13. A magnetic resonance eye imaging system according to the previous item, the magnetic resonance eye imaging system further comprising
   an image constructing arrangement adapted to
      bin the acquired magnetic resonance image data into groups according to eye orientation information data;
         and
      to construct a magnetic resonance image eye image from a selection of groups of magnetic resonance image data.
14. A magnetic resonance eye image construction arrangement for constructing eye images from magnetic resonance imaging data acquired during movement of the eye,
   the eye image construction arrangement comprising
      an input
      for inputting magnetic resonance image data acquired from a region of interest including the eye and while the eye is moving,
      and
      for inputting eye orientation information data relating to eye orientation information data determined during magnetic resonance image data acquisition,
   and an image constructing arrangement adapted to
      bin the acquired magnetic resonance image data into groups according to eye orientation information data;
      and
      to construct a magnetic resonance image eye image from a selection of groups of magnetic resonance image data.

The invention will now be described by way of example only with respect to the drawing. In the drawing,

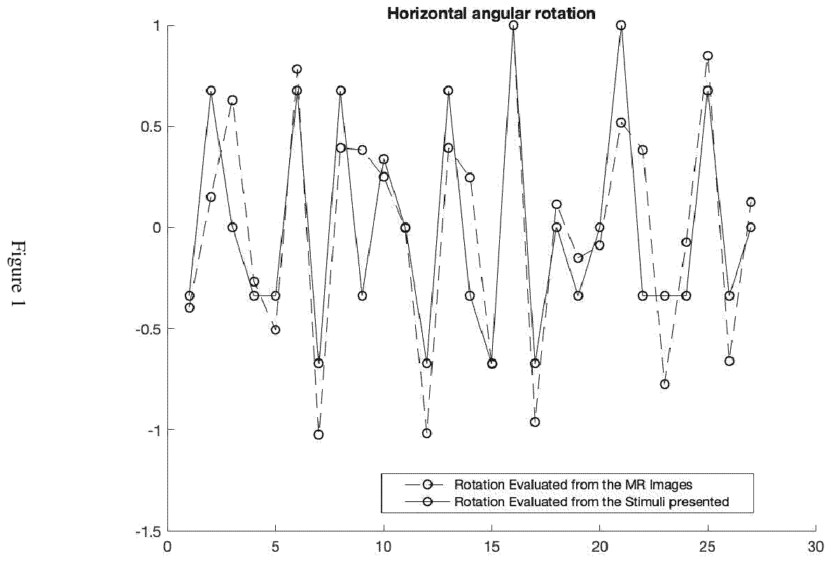

FIG. 1 represents a comparison between the horizontal angular orientation of the Eye determined from the reconstructed images and the orientation according to the eye tracker used in the experimental setup;

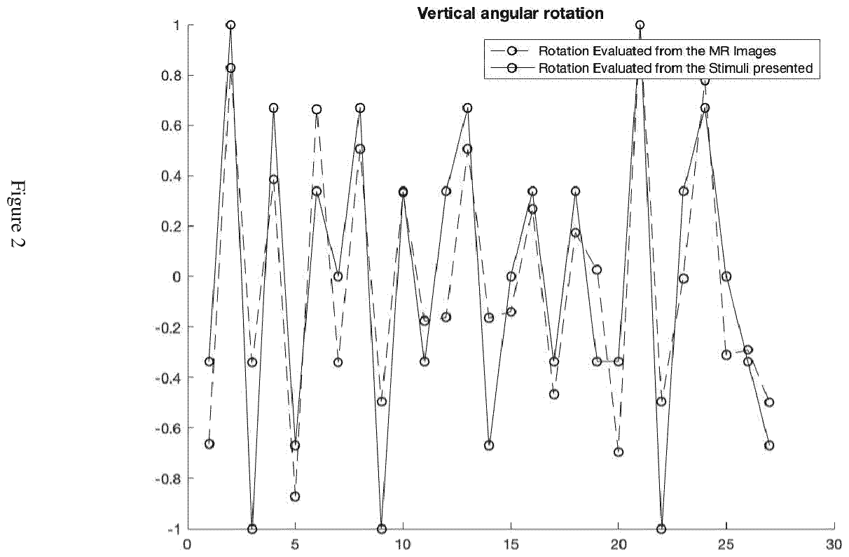

FIG. 2 represents a comparison between the vertical angular orientation of the Eye determined from the reconstructed images and the orientation according to the eye tracker used in the experimental setup;

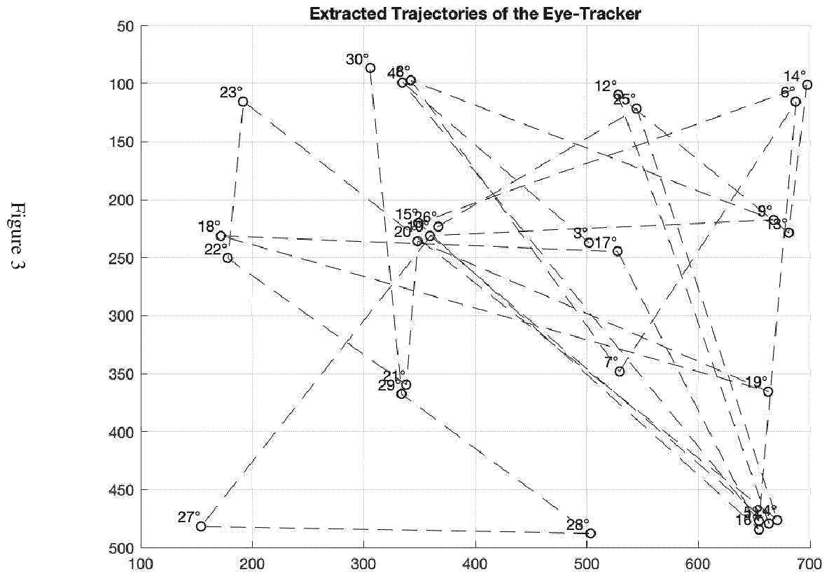

FIG. 3 represents trajectories determined with the Eye-Tracker;

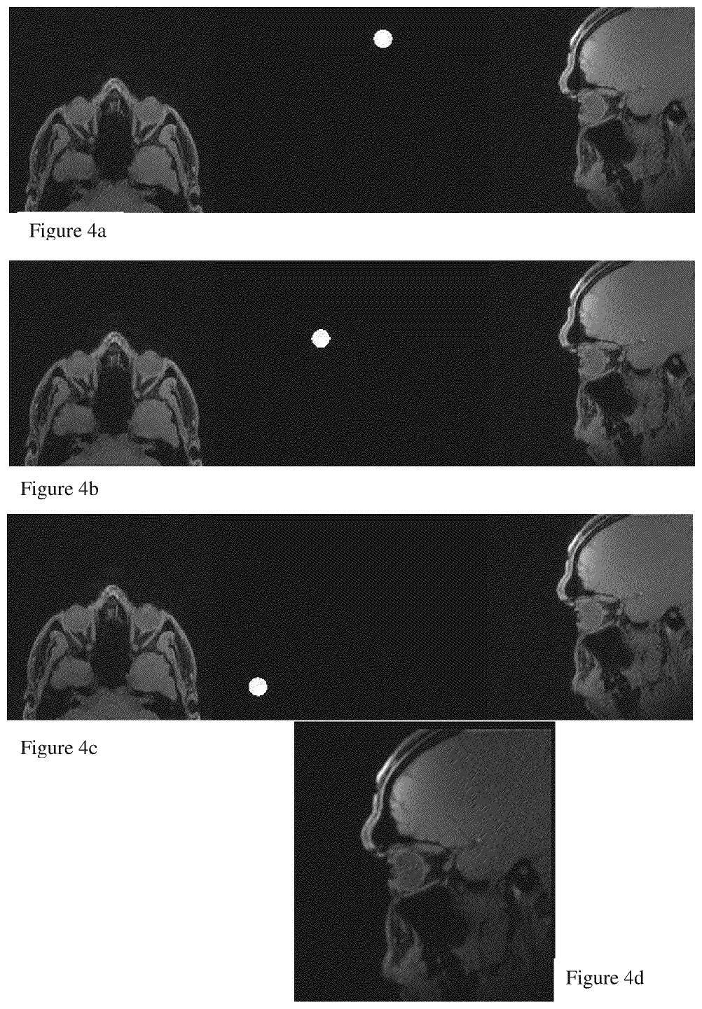

FIG. 4 a 2D eye images obtained from example 1 for two different sections through the head with the white point showing the direction into which the test person is looking;

FIG. 4 b same as FIG. 4a, but with the test person looking into another direction;

FIG. 4 c same as FIG. 4a, but with the test person looking into yet another direction;

FIG. 4 d an enlarged part of one of the sections through the head shown in FIG. 4 a-c;

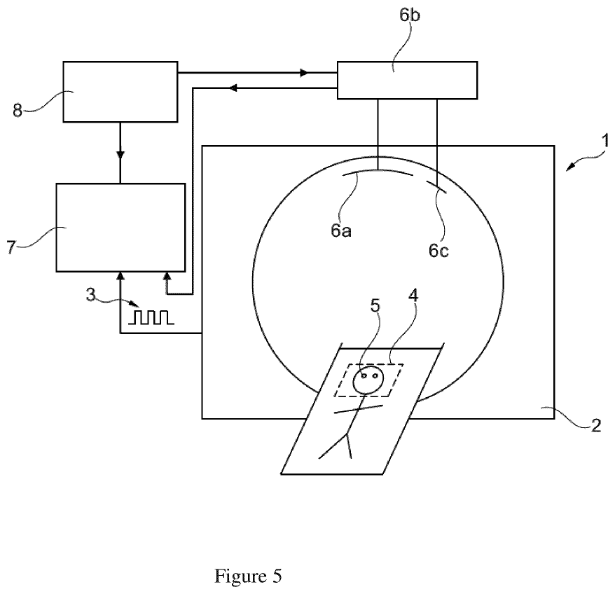

FIG. 5 a magnetic resonance eye imaging system according to the invention.

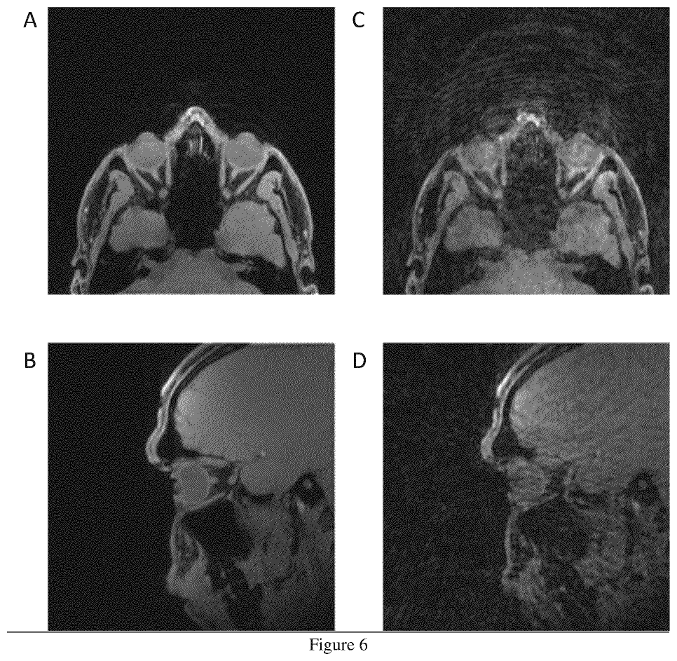

FIG. 6 represents a comparison between an image reconstructed according to the method of the present invention, and that reconstructed using the same amount of data collected in a consecutive period of time.

In FIG. 5, reference numeral 1 generally refers to a magnetic resonance eye imaging system 1 comprising a magnetic resonance image data acquisition arrangement 2 adapted to acquire magnetic resonance image data 3 from a region of interest 4 including the eye 5 and while the eye is moving, and eye orientation information data determination arrangement 6 adapted for determining eye orientation information data during magnetic resonance image data acquisition in a manner allowing to assign an orientation of the eye to different parts of the magnetic resonance image data. The magnetic resonance eye imaging system 1 also comprises an image constructing arrangement 7 adapted to bin the acquired magnetic resonance image data into groups according to eye orientation information data and to construct a magnetic resonance image eye image from a selection of groups of magnetic resonance image data.

Note that although in FIG. 5, the image constructing arrangement 7 is shown in close proximity to the magnetic resonance image data acquisition arrangement 2, it would be well possible to space the image constructing arrangement 7 far apart from the magnetic resonance image data acquisition arrangement 2. In particular, it would be possible to acquire the data in a medical practice and communicate the data to a remote center for analysis and/or diagnosis.

The magnetic resonance image data acquisition arrangement 2 shown in FIG. 5 can be based on a commercially available device. In a practical embodiment, a standard MAGNETOM Prismafit 3T clinical MRI scanner by Siemens Healthcare AG was used as a magnetic resonance image data acquisition arrangement 2. This MRI scanner can be operated using a number of different definable pulse sequences and with different receiving antenna coils; in the practical embodiment, an antenna coil arrangement was used adapted for skull imaging. The signals received with the antenna coils will vary over time in a manner depending from both the excitation pulses used and the anatomical details of the person examined; the signals are conditioned e.g. amplified appropriately and then digitized using conventional suitable circuitry so that magnetic resonance image data 3 is acquired from which by proper magnetic resonance image data processing in an image constructing arrangement 7 a magnetic resonance image eye image can be constructed. Accordingly, the magnetic resonance image data acquisition arrangement 2 was adapted to acquire magnetic resonance image data 3 from a region of interest 4 including the eye.

Furthermore, in a practical implementation, the MAGNETOM Prismafit 3T clinical MRI scanner by Siemens Healthcare AG used as a magnetic resonance image data acquisition arrangement 2 is adapted to generate an uninterrupted gradient recalled echo (GRE) sequence with lipid-insensitive binomial off-resonant RF excitation (LIBRE) for fat suppression was applied and the acquisition used a 3D radial phyllotaxis sampling pattern with spiral trajectories rotated by the golden-angle for uniform k-space coverage over a field-of view of (192 mm)3 with 1 mm3 isotropic resolution.

Within the tube of the a magnetic resonance image data acquisition arrangement 2, a display 6a constituting a part of the eye orientation information data determination arrangement 6 is placed capable of showing to a person examined a white circle on a black background at different positions. The size of the display is selected such that the person examined has to look up, down, left and right respectively when the white circle is shown close to the border of the display. In a practical embodiment, the display can be controlled by a programmable computer 6b in a manner such that changing images as changing stimuli to the patient can be shown that each have a duration of e.g. 5 seconds. (For the record: such duration is not limiting and other durations are obviously possible; also note that rather than using a separate computer 6b, the hardware of e.g. the image constructing arrangement 7 could also be used where this is a computer). For each distinct visual stimulus, the white circle was shown at a different position. In a practical embodiment the computer can be programmed such that each stimulus was repeated 6 times during an examination for a total of 96 trials opportunely randomized.

Furthermore, a commercial eye-tracker 6c constituting a further part of the eye orientation information data determination arrangement 6 is placed in the tube of the magnetic resonance image data acquisition arrangement 2, the eye-tracker 6c being arranged for observing the direction to which the person examined is looking during operation of the as magnetic resonance image data acquisition arrangement 2. In a practical implementation, an eye tracker EyeLink 1000Plus eye-tracking system has been used. The eye tracker was operated in parallel to the generation of the uninterrupted gradient recalled echo (GRE) sequence and a Syncbox 8 by NordicNeuroLab was provided to synchronize the measurements with the MRI scanner, i.e time stamps for both the eye orientation information data and the magnetic resonance image data 3 are generated by Syncbox 8.

### EXAMPLE 1

For healthy adult volunteers, magnetic resonance image data were acquired using a standard MAGNETOM Prismafit 3T clinical MRI scanner by Siemens Healthcare AG.

An uninterrupted gradient recalled echo (GRE) sequence with lipid-insensitive binomial off-resonant RF excitation (LIBRE) for fat suppression was applied and the acquisition used a 3D radial sampling pattern rotated by the golden-angle for uniform k-space coverage. The field-of view was (192 mm)³ with 1 mm³ isotropic resolution.

During acquisition of magnetic resonance image data, sixteen distinct visual stimuli were randomly presented six times to each volunteer.

Each stimulus had a duration of 5 seconds and consisted of a white circle on a black background; for each distinct visual stimulus, the white circle was shown at a different position. Each stimulus was repeated 6 times during the experiment for a total of 96 trials opportunely randomized to ensure uniform sampling distribution of the readouts in k-space.

Simultaneous with the presentation of the sixteen distinct visual stimuli, eye movements were tracked using an Eye-tracker EyeLink 1000Plus eye-tracking system that was synchronized with the MRI scanner via a Syncbox by NordicNeuroLab. An example of the trajectories extracted with the Eye-Tracker is shown in FIG. 3.

The post-processed Eye-tracker data were then used for binning the data obtained during the time interval spent in a given orientation state and for matching the k-space readouts corresponding to the same stimulus presentation.

Orientation-resolved 5D image reconstruction (x-y-z-α-β dimensions, where α and β represent the angular displacement of the eye in the up-down and left-right directions) was performed using a k-t sparse SENSE algorithm that exploits sparsity both along the α and β directions.

For all volunteers, 3D orientation-resolved images of the eye with 1 mm³ isotropic resolution could be successfully acquired and reconstructed. Despite the fact that each stimulus had a 5 second duration which is long compared to fast movements of the eye occurring sometimes during prolonged observations of a target, the images were void of orientation artifacts and eye orientations across the presentation of the different visual stimuli were clearly reconstructed (FIG. 4).

It was found that the horizontal angular orientation of the Eye deduced from the reconstructed Images corresponds closely to the determination of eye orientation based on the eye tracker, cmp. FIG. 1.

Furthermore, it was also found that the angular orientation of the Eye deduced from the reconstructed Images corresponds closely to the determination of eye orientation based on the eye tracker, cmp. FIG. 2.

Magnetic resonance images obtained in this manner are shown in FIG. 4 a-c for three different orientations. FIG. 4d depicts an enlarged view of a section as shown in FIG. 4a-c.

It can be concluded that the proposed method allows to obtain high quality orientation resolved eye images using a free running, uninterrupted MR excitation sequence and additional eye orientation information data.

As will be obvious from the above description, the present invention thus allows to reconstruct magnetic resonance images of an object while moving. It is inter alia suggested in one embodiment to provide magnetic resonance eye images based on a known pattern to be followed; accordingly, a stimulation protocol is implemented leading to a stimulated eye orientation. However, not only is in a preferred embodiment a suitable stimulation protocol implemented, but also the data acquired are treated in a specific manner overcoming limitations of prior part ophtalmic technologies requiring anesthesia.

### EXAMPLE 2

Images were acquired using a 3T clinical MRI scanner (MAGNETOM Prisma^{fit}, Siemens Healthcare AG) with a 22-channel head coil, using a prototype uninterrupted gradient recalled echo (GRE) sequence with lipid-insensitive binomial off-resonant RF excitation (LIBRE) for fat suppression. The acquisition used a 3D radial sampling pattern, the spiral phyllotaxis trajectory where each interleaf is rotated by the golden-angle to allow uniform k-space coverage. Eye movements were tracked using an eye-tracking system (EyeLink 1000Plus, SR Research) synchronized with the MRI scanner via Syncbox (NordicNeuroLab). An Experiment builder (EyeLink) program was developed and used to control the calibration of the Eye-Tracker from outside the scanner room and to correctly synchronize the different hardware components of the experiment. Eye-tracked trajectories, together with related trial number and temporal synchronization information, were extracted from the eye-tracking software. The right eye was the one tracked during the acquisition. Eye movement trajectories were recorded using infrared, with a sampling rate of 2000 Hz, through a mirror positioned inside the scanner bore, replacing the standard head-coil mirror usually available, which is not infrared compatible. The FoV was 192 mm³ with 1 mm³ isotropic resolution, TR/TE=6.4/2.94 ms, receiver bandwidth BW=501 Hz/px, and radiofrequency excitation angle FA=5°. The stimulation protocol was divided into 3 distinct phases, all consisting of a grey circle positioned at specific locations on a black background. These circular stimuli guided the eye movements. First, an initial period of fixation was performed, where the image presented to the participant was the static grey circle positioned at the centre of the screen. This first part of the experiment allowed for performing the sequence localizer while the eye was in a static position. Second, 96 visual stimuli were presented to each participant. Each stimulus corresponded to one among 16 different locations the grey circle on a 4×4 grid.

Each presentation had a duration of 5 seconds and was repeated 6 times in distinct and randomized moments during the experiment. This part of the acquisition lasted for 8 minutes in total. Third, the fixation circle was presented again, as in the first part of the experiment, to conclude the acquisition. The presentations during the second phase of the experiment were opportunely randomized to ensure a uniform sampling distribution of the readouts in k-space during the following retrospective motion-resolved reconstruction step. A total of 81906 readout profiles, divided into 3723 interleaves, were acquired.

The continuously acquired data, as enabled by the free-running approach to data collection, can be arbitrarily partitioned into different bins thanks to the golden-angle distribution properties. The processed eye-tracker data were used to bin the time intervals of each motion state and to match the k-space readouts corresponding to the same stimulus presentation, hence leading to the same motion-resolved 3D image. Motion-resolved 5D image reconstruction (x-y-z-α-β dimensions, where α and β represent the eye angular rotations in the horizontal and vertical directions, respectively) was performed using a k-t sparse SENSE algorithm (image under-sampling 8.8%), exploiting sparsity both along the α and β directions. The values of α and β are deduced from the eye-tracker recordings and correspond to those determined from the reconstructed images, once normalized. For one selected subject and eye position, a typical reconstructed image is shown in FIG. 6 in panels A and B.

### REFERENCE EXAMPLE 2

The dataset of Example 1 is used in Reference Example 2, wherein no binning according to eye orientation information data is performed. Instead of performing a 5D k-t sparse SENSE reconstruction, we perform a 4D reconstruction (NO k-t sparse SENSE) having the time t as fourth dimension. The sections shown on the right are composed by readouts acquired continuously for 30 s, matching the bin size of the previous compressed sensing reconstruction. The resulting reconstruction is shown in FIG. 6 panel C and D.

As it can be seen, from comparing panels A and C, as well as B and D in FIG. 6, binning of MRI data according to eye orientation information data allows for reconstructions that are less blurred and comprise higher level of detail.

1. A magnetic resonance eye imaging method, wherein an eye image is obtained from magnetic resonance image data acquired while the eye is moving, comprising
determining eye orientation information data during magnetic resonance image data acquisition;
binning the acquired magnetic resonance image data into groups according to eye orientation information data;
and
constructing a magnetic resonance image eye image from a selection of groups of magnetic resonance image data.

2. The magnetic resonance eye imaging method according to claim 1, wherein the magnetic resonance image data are acquired with a free running magnetic resonance image and/or in a manner not triggered by an eye orientation determined.

3. The magnetic resonance eye imaging method according to claim 1, wherein the eye image is obtained from magnetic resonance image data acquired intermittent to or simultaneous with an eye motion.

4. The magnetic resonance eye imaging method according to claim 1, wherein determining eye orientation information data during magnetic resonance image data acquisition comprises tracking the orientation of the eye or the orientation of a surface related to the eye.

5. The magnetic resonance eye imaging method according to claim 1, wherein determining eye orientation information data during magnetic resonance image data acquisition comprises causing the eye to orient in space according to a known pattern.

6. The magnetic resonance eye imaging method according to claim 1, wherein determining eye orientation information data during magnetic resonance image data acquisition comprises determination of eye orientation information data according to a two-dimensional pattern.

7. The magnetic resonance eye imaging method according to claim 1, wherein binning the acquired magnetic resonance image data into groups according to eye orientation information data comprises a two-dimensional binning.

8. The magnetic resonance eye imaging method according to claim 1, wherein constructing a magnetic resonance image from a selection of groups of magnetic resonance image data comprises constructing a 3D image having a number of planes.

9. The magnetic resonance eye imaging method according to claim 1, wherein constructing a magnetic resonance eye image from a selection of groups of magnetic resonance image data comprises constructing a sequence of images constructed according to a sequence of orientations.

10. The magnetic resonance eye imaging method according to claim 1, wherein a body part is scanned comprising the entire visceral cavity wherein the eye is located.

11. The magnetic resonance eye imaging method according to claim 1, wherein the eye orientation is determined by a showing a pattern to be followed.

12. A magnetic resonance eye imaging system,
comprising
a magnetic resonance image data acquisition arrangement adapted to acquire magnetic resonance image data from a region of interest including the eye and while the eye is moving,
and
an eye orientation information data determination arrangement
adapted for determining eye orientation information data during magnetic resonance image data acquisition
in a manner allowing to assign an orientation of the eye to different parts of the magnetic resonance image data.

13. The magnetic resonance eye imaging system according to claim 12, the magnetic resonance eye imaging system further comprising
an image constructing arrangement adapted to
bin the acquired magnetic resonance image data into groups according to eye orientation information data;
and
to construct a magnetic resonance image eye image from a selection of groups of magnetic resonance image data.

14. A magnetic resonance eye image construction arrangement for constructing eye images from magnetic resonance imaging data acquired during movement of the eye,
the eye image construction arrangement comprising
an input
for inputting magnetic resonance image data acquired from a region of interest including the eye and while the eye is moving,
and
for inputting eye orientation information data relating to eye orientation information data determined during magnetic resonance image data acquisition,
and an image constructing arrangement adapted to
bin the acquired magnetic resonance image data into groups according to eye orientation information data;
and
to construct a magnetic resonance image eye image from a selection of groups of magnetic resonance image data.
